(19) United States
(12) Patent Application Publication
(10) Pub. No.: US 2026/0027358 A1
(43) Pub. Date: Jan. 29, 2026

(54) BIOIMPEDANCE-BASED SYSTEMS AND METHODS FOR TREATING BLADDER AND/OR BOWEL DYSFUNCTION

(71) Applicant: NSPIRE MEDICAL SYSTEMS, INC., Golden Valley, MN (US)

(72) Inventors: Stephen Lorne Bolea, Excelsior, MN (US); Christopher Poletto, North Oaks, MN (US); John Rondoni, Plymouth, MN (US)

(73) Assignee: NSPIRE MEDICAL SYSTEMS, INC., Golden Valley, MN (US)

(21) Appl. No.: 19/144,232

(22) PCT Filed: Dec. 29, 2023
(86) PCT No.: PCT/US2023/086429

§ 371 Date: Jun. 27, 2025

Related U.S. Application Data
(60) Provisional application No. 63/436,502, filed on Dec. 31, 2022.

(51) Int. Cl. A61N 1/36; A61B 5/053; A61B 5/20

(52) U.S. Cl. CPC A61N 1/36007; A61B 5/053; A61B 5/20

(57) ABSTRACT

Systems and methods for treating bladder and/or bowel dysfunction of a patient includes a stimulation element implanted to stimulate one or more target sites, and a bioimpedance sensor to sense at least one bioimpedance parameter of the patient. In some examples. stimulation energy is applied to an anatomical structure of the patient as a function of the sensed bioimpedance parameter. for example to address the potential bladder or bowel dysfunction event.

A portion of the population suffers from bladder and/or bowel dysfunction, such as one or both of urinary incontinence (or bladder incontinence) and fecal incontinence (or bowel incontinence). Diet, training, slings, and drug therapies may fail to treat incontinence.

### BRIEF DESCRIPTION OF DRAWINGS

FIG. 1 is a schematic illustration of anatomy of a human pelvic region.

FIG. 2 is a schematic illustration of the pelvic region of FIG. 1 and various nerves.

FIG. 3 is a block diagram of a treatment system in accordance with principles of the present disclosure.

FIG. 4 is a simplified perspective view of a treatment system in accordance with principles of the present disclosure applied to anatomy of a patient.

FIG. 5 is a simplified top sectional view of a treatment system in accordance with principles of the present disclosure applied to anatomy of a patient.

FIG. 6 is a simplified front view of a treatment system in accordance with principles of the present disclosure applied to anatomy of a patient.

FIG. 7 is a diagram of portions of the human anatomy including the bladder, and identifying possible implant locations of system components of the present disclosure.

FIG. 8 is a simplified perspective view of a treatment system in accordance with principles of the present disclosure applied to anatomy of a patient.

FIG. 9 is a block diagram schematically representing a care engine of a control portion.

FIG. 10 is a diagram schematically representing a patient's body, implantable components, and/or external elements of an example device and/or for use in an example method.

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure. The following detailed description, therefore, is not to be taken in a limiting sense. It is to be understood that features of the various examples described herein may be combined, in part or whole, with each other, unless specifically noted otherwise.

At least some examples of the present disclosure are directed to implantable devices for diagnosis, therapy, and/or other care of medical conditions. At least some examples may comprise implantable devices and/or methods of implanting devices useful for treating bladder or bowel dysfunctions, including one or both of urinary incontinence and fecal incontinence of a patient, or other pelvic disorders. At least some such examples comprise implanting an electrode to deliver a nerve-stimulation signal to one or more nerves or nerve branches to activate a corresponding external sphincter, such as a branch of the pudendal nerve that activates the external urethral sphincter and/or the external anal sphincter. In some embodiments, operation of the implantable device is controlled in response to sensed information of the patient.

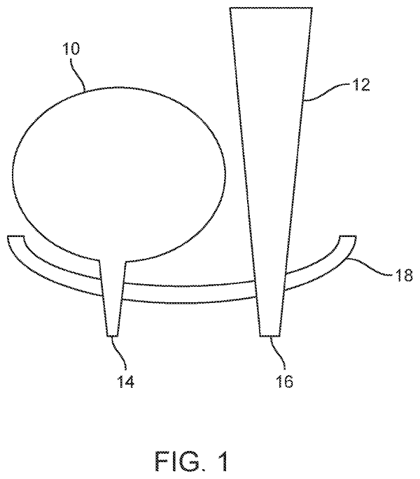

With reference to the greatly simplified view of FIG. 1, the human pelvic region includes a bladder 10 and a rectum 12. Contents of the bladder 10 are evacuated through a urethra 14, whereas contents of the rectum 12 are evacuated through the anus 16. Pelvic floor muscles 18 support the pelvic organs and span the bottom of the pelvis. The pelvic floor muscle layer 18 has holes for passage of the urethra 14 and the anus 16, and normally wraps quite firmly around these holes to help keep the passages shut.

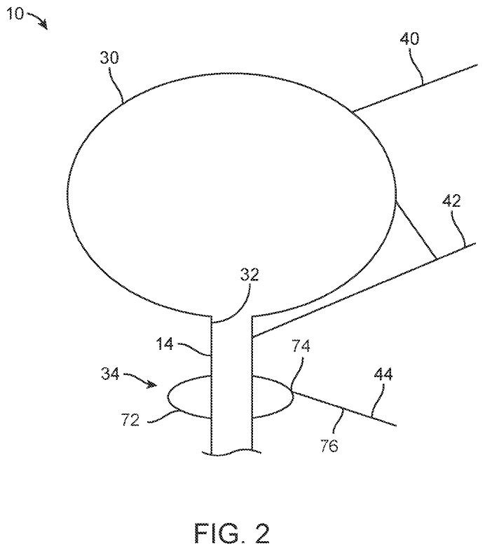

With additional references to the greatly simplified view of FIG. 2, the bladder 10 is a hollow muscular organ connected to the kidneys by the ureters. The detrusor 30 muscle (referenced generally) is smooth muscle found in the wall of the bladder 10. The urethra 14 is a tube or duct by which urine is conveyed out of the body from the bladder 10. Internal and external sphincters control flow of urine through the urethra 14; under normal conditions, when either of these muscles contracts, the urethra 14 is sealed shut. In particular, an internal urethral sphincter (IUS) 32 (referenced generally) is a smooth muscle that constricts the internal orifice of the urethra 14. The IUS 32 is located at the junction of the urethra 14 with the bladder 10 and is continuous with the detrusor muscle 30, but is anatomically and functionally fully independent from the detrusor muscle 30. An external urethral sphincter (EUS) 34 is located in the deep perineal pouch, at the bladder's 10 distal inferior end around the mid urethra in females and inferior to the prostate in males. Urine is excreted from the kidneys and stored in the bladder 10 before elimination via the urethra 14 during what is known as the micturition reflex. During periods of bladder filling, the storage of urine is promoted by the actions of the internal and external urethral sphincters 32, 34 and the pelvic floor musculature 18. During micturition, these sphincters 32, 34 relax and the smooth muscle of the bladder (the detrusor muscle 30) contracts, resulting in the expulsion of urine.

The body of the bladder 10 is directly innervated by efferent fibers that arise from parasympathetic postganglionic neurons in the pelvic ganglia and intramural ganglia and by efferent fibers that arise from sympathetic postganglionic neurons in the lumbosacral sympathetic chain and hypogastric ganglia/pelvic ganglia. This is generally reflected in FIG. 2 by reference to a pelvic nerve 40 and a hypogastric nerve 42. The internal urethral sphincter 32 receives innervation from the hypogastric nerve 42. The external urethral sphincter 34 is directly innervated by motor neurons in the sacral segments of the spinal cord via the pudendal nerve 44.

Urinary continence is generally defined as the act of storing urine in the bladder 10 until the bladder 10 can be appropriately evacuated. Urinary continence requires control of the detrusor muscle 30 and is the result of complex coordination between multiple centers in the brain, brain stem, spinal cord, and peripheral nerves. As described above, micturition is a coordinated act of bladder elimination that involves relaxing the pelvic floor muscles 18, contracting the detrusor muscle 30, and simultaneously opening the urethral sphincters 32, 34 to achieve complete emptying of the bladder. Stress incontinence can be defined as the involuntary leakage of urine from the bladder 10 accompanying physical activity (e.g., laughing, coughing, sneezing, etc.) which places increased pressure on the abdomen. The leakage occurs even though the bladder muscles (detrusor muscle 30) is not contracting and an urge to urinate is not present. Stress incontinence can develop when the urethral sphincters 32, 34, the pelvic floor muscles 18, or all of these structures have been weakened or damaged and cannot dependably hold in urine. With urethral hypermobility, the bladder 10 and urethra 14 shift downward when abdominal pressure rises, and there is no hammock-like support for the urethra 14 to be compressed against to keep it closed. With urethral incompetence, problems in the urinary sphincter 32, 34 keep it from closing fully or allow it to pop open under pressure. Urinary urge incontinence (“UUI”) (sometimes referred to as overactive bladder (“OAB”) or detrusor overactivity) entails the involuntary leakage of urine from the bladder 10 when a sudden strong need to urinate is felt. There is a sudden involuntary contraction of the muscular wall (the detrusor 30) of the bladder that signals an immediate need to urinate, which can happen even when the bladder 10 is not full. Mixed incontinence is the term used to a combination of both overactive bladder and stress incontinence.

Internal and external sphincters are similarly provided with the anus 16 (i.e., the internal anal sphincter and the external anal sphincter), acting to keep the anal canal and orifice closed. Action of the internal anal sphincter (IAS) is entirely involuntary, and it is in a state of continuous maximal contraction. The external anal sphincter (EAS) is always in a state of contraction, but can be voluntarily put into a condition of greater contraction so as to more firmly occlude the anal orifice. Similar to urinary continence, bowel continence is the act of storing feces until an acceptable time and opportunity for elimination. Bowel continence requires competent internal and external sphincters, pelvic floor musculature, and intact neurological pathways. Neurological control of bowel continence is complex and requires coordinated reflex activities from the autonomic and enteric nervous systems. The colon can be visualized as a closed, pliant tube bounded by the ileocecal valve and the anal sphincter. The continuous, smooth muscle layer at the end of the rectum 12 thickens to form the internal anal sphincter (IAS); the external anal sphincter (EAS) is a circular band of striated muscle that contracts with the pelvic floor. Parasympathetic stimulation of the IAS from the pelvic plexus originates from the sacral cord (S1 to S2). Sympathetic stimulation of the IAS causes contraction. The EAS is composed of both smooth and striated muscle. The smooth muscle of the EAS is innervated by the enteric nervous system. The striated component of the EAS is innervated by the pudendal nerve that exits the cord at sacral levels S2, S3, and S4.

Fecal incontinence can be defined as the involuntary loss of rectal contents (feces, gas) through the anal canal and the inability to postpone an evacuation until socially convenient. For example, injuries to one or both of the EAS and IAS may make it difficult to hold stool back properly. Injury to the nerves that sense stool in the rectum or those that control the anal sphincter can also lead to fecal incontinence. A generalized weakness of the pelvic floor 18 can lead to an impaired barrier to stool in the rectum 12 entering the anal canal, and this is associated with incontinence to solids. The pelvic floor 18 is innervated by the pudendal nerve and the S3 and S4 branches of the pelvic plexus. If the pelvic floor muscles 18 lose their innervation, they cease to contract and their muscle fibers are in time replaced by fibrous tissue, which is associated with pelvic floor weakness and incontinence.

With the above in mind, various treatment systems and methods have been disclosed that treat bladder and/or bowel dysfunction (e.g., one or more of urinary incontinence, UUI and fecal incontinence) by supplying stimulation signals to an electrode implanted to apply the stimulation signal to one or more nerves and/or muscles of the patient that, for example, influence the behavior of musculature of the pelvic region of the patient, for example musculature relating to one or both of urinary incontinence and fecal incontinence (e.g., the external urethral sphincter 34, the internal urethral sphincter 32, pelvic floor muscles 18, the external anal sphincter, the internal anal sphincter, etc.). Examples of such systems and methods are provided in PCT Publication No. 2020/243104 (Rondoni, et al.) and PCT Publication No. WO 2022/192726 (Rondoni, et al.) the entire teachings of each of which are incorporated herein by reference.

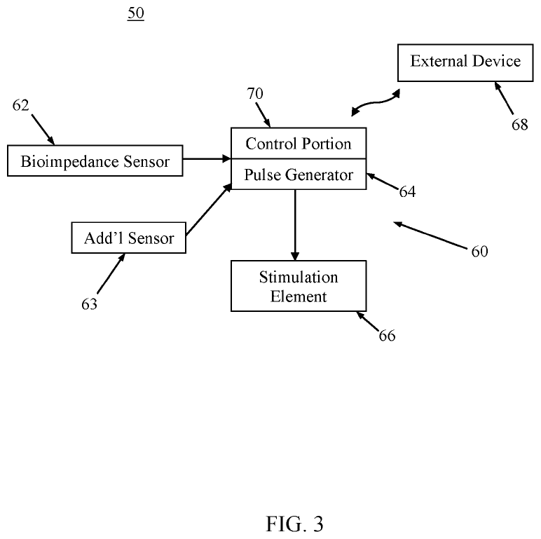

One example of a treatment system 50 for treatment of bladder and/or bowel dysfunction in accordance with principles of the present disclosure is provided in FIG. 3 and includes an implantable medical device (IMD) 60 (referenced generally), one or more bioimpedance sensors 62, and optionally one or more additional sensors 63 that may or may not be a bioimpedance sensor (e.g., the additional sensor(s) 63, where provided, can include one or more of an accelerometer, a pressure sensor, a strain sensor, etc.). In general terms, the IMD 60 includes an implantable pulse generator or implantable component of a pulse generator (collectively identified as “IPG”) 64 and one or more stimulation elements (e.g., electrode or electrode assembly) 66. The IPG 64 is configured for implantation into a patient, and is configured to provide and/or assist in the performance of therapy to the patient. With formats in which the IPG 64 is an implantable pulse generator, a power source (e.g., battery) is carried within a housing of the implantable pulse generator and from which stimulation energy is generated. With formats in which the IPG 64 is an implantable component of a pulse generator, the implantable component(s) can include a receiver unit (e.g., receiver coil or similar device) that receives a signal from an external device (external the patient) that typically would be positioned on top of the skin over the location of the receiver coil. The external device can generate/deliver the stimulation energy at desired setting (e.g., amplitude, pulse width, frequency, pulse train length, etc.) to be received by the implanted receiver unit and conducted to the stimulation element(s) 66 for activation of tissue. The implanted receiver unit may or may not operate to modify the signal it receives prior to delivery to the stimulation element(s) 66. The external transmitter/controller may receive sensing signals from external sensor, receive sensing signals from the implanted portion of the implantable component via telemetry, etc. Unless stated otherwise, reference to “IPG 64” is inclusive of both an implantable pulse generator and an implantable component of a pulse generator as described above. The stimulation element 66 is configured to be implanted proximate a selected segment or region of the patient's anatomy, and is electrically connected to the IPG 64. In other embodiments, the IPG 64 and the stimulation element 66 can be provided as components of a single or integral device, such as a microstimulator, as are known in the art. The IPG 64 is programmed to deliver (or is prompted to deliver) stimulation signals to the stimulation element 66 that in turn apply the signal. In some embodiments, the IPG 64 is programmed (or is prompted) to initiate, cease and/or modulate (e.g., titrate) delivered stimulation signals based upon one or more physical parameters of the patient. In this regard, the sensor(s) 62, 63 sense the physical parameter of interest, and signal the so-sensed parameter to the IPG 64 (or other component controlling operation of the IPG 64). The sensor(s) 62, 63 can be carried by the IPG 64, can be connected to the IPG 64, or can be a standalone component not physically connected to the IPG 64. The sensor(s) 62, 63 can be self-contained, and communicate with the IPG 64 in some optional embodiments. In some embodiments, the sensors 62, 63, the IPG 64, and the stimulation element 66 can be provided as components of a single or integral device. In some embodiments, the treatment system 50 can further include an optional external device 68. Where provided, the external device 68 can, in some non-limiting embodiments, wirelessly communicate with the IMD 60.

The IPG 64 can assume various forms known in the art for generating a nerve-stimulating signal for delivery to the stimulation element(s) 66. For example, the IPG 64 can include a sealed case or enclosure maintaining a power source (e.g., battery) and electrical/circuitry components appropriate for formatting energy from the power source as the desired stimulation signal (e.g., a nerve-stimulation signal). In some embodiments, the IPG 64 as provided as part of, or is electronically linked to, a control system that includes a control portion 70 providing one example implementation of a control portion forming a part of, implementing, and/or generally managing stimulation element(s), power/control elements (e.g. pulse generators, microstimulators), sensors, and related elements, devices, user interfaces, instructions, information, engines, elements, functions, actions, and/or methods, as described throughout examples of the present disclosure. In some examples, the control portion 70 includes a controller and a memory. In general terms, the controller comprises at least one processor and associated memories. The controller is electrically couplable to, and in communication with, memory to generate control signals to direct operation of at least some of the stimulation elements, power/control elements (e.g., pulse generators, microstimulators) sensors, and related elements, devices, user interfaces, instructions, information, engines, elements, functions, actions, and/or methods, as described throughout examples of the present disclosure. In some non-limiting examples, these generated control signals include, but are not limited to, employing instructions and/or information stored in the memory to at least direct and manage treatment of bladder and/or bowel dysfunction by stimulating nerve(s), nerve branch(es) and/or muscle(s), for example to activate one or more of the external urethral sphincter 34 and the external anal sphincter, and/or pelvic floor nerves (e.g., the pudendal nerve 44, the sacral nerve) to relax the detrusor muscle 30 and prevent or reduce urgency or frequency.

In some instances, the controller or control portion 70 may sometimes be referred to as being programmed to perform the actions, functions, routines, etc. of the present disclosure. In some examples, at least some of the stored instructions are implemented as, or may be referred to as, a care engine, a sensing engine, monitoring engine, and/or treatment engine. In some examples, at least some of the stored instructions and/or information may form at least part of, and/or, may be referred to as a care engine, sensing engine, monitoring engine, and/or treatment engine.

In response to or based upon commands received via a user interface and/or via machine readable instructions, the controller generates control signals as described above in accordance with at least some of the examples of the present disclosure. In some examples, the controller is embodied in a general purpose computing device while in some examples, the controller is incorporated into or associated with at least some of the stimulation elements, power/control elements (e.g. pulse generators, microstimulators), sensors, and related elements, devices, user interfaces, instructions, information, engines, functions, actions, and/or method, etc. as described throughout examples of the present disclosure.

For purposes of the present disclosure, in reference to the controller, the term “processor” shall mean a presently developed or future developed processor (or processing resources) that executes machine readable instructions contained in a memory. In some examples, execution of the machine readable instructions, such as those provided via the memory of the control portion 70 cause the processor to perform the above-identified actions, such as operating the controller to implement the sensing, monitoring, treatment, etc. as generally described in (or consistent with) at least some examples of the present disclosure. The machine readable instructions may be loaded in a random access memory (RAM) for execution by the processor from their stored location in a read only memory (ROM), a mass storage device, or some other persistent storage (e.g., non-transitory tangible medium or non-volatile tangible medium), as represented by the memory. In some examples, the machine readable instructions may comprise a sequence of instructions, a processor-executable machine learning model, or the like. In some examples, the memory comprises a computer readable tangible medium providing non-volatile storage of the machine readable instructions executable by a process of the controller. In some examples, the computer readable tangible medium may sometimes be referred to as, and/or comprise at least a portion of, a computer program product. In other examples, hard wired circuitry may be used in place of or in combination with machine readable instructions to implement the functions described. For example, the controller may be embodied as part of at least one application-specific integrated circuit (ASIC), at least one field-programmable gate array (FPGA), and/or the like. In at least some examples, the controller is not limited to any specific combination of hardware circuitry and machine readable instructions, nor limited to any particular source for the machine readable instructions executed by the controller.

In some examples, the control portion 70 may be entirely implemented within or by a stand-alone device.

In some examples, the control portion 70 may be partially implemented in the IPG 64 and partially implemented in a computing resource separate from, and independent of, the IPG 64. For instance, in some examples the control portion 70 may be implemented via a server accessible via the cloud and/or other network pathways. In some examples, the control portion 70 may be distributed or apportioned among multiple devices or resources such as among a server, a neurostimulation or neuromodulation treatment device (or portion thereof), and/or a user interface.

In some examples, the control portion 70 is entirely implemented within or by the IPG 64 (thereby defining an IPG assembly), which has at least some of substantially the same features and attributes as a pulse generator (e.g., power/control element, microstimulator) as described throughout the present disclosure. In some examples, the control portion 70 is entirely implemented within or by a remote control (e.g., a programmer) external to the patient's body, such as a patient control and/or a physician control (e.g., the external device 68). In some examples, the control portion 70 is partially implemented in the IPG 64 assembly and partially implemented in the remote control (at least one of the patient control and the physician control).

The systems and methods of the present disclosure are in no way limited to a particular stimulation target site(s) or a particular stimulation therapy regimen. The stimulation therapies or algorithms programmed to, or implemented by, the control portion 70 can be of any format deemed useful for the patient being treated, and may or may not act upon information from the optional, additional sensor(s) 63. With reference between FIGS. 1-3, the system 50 can be configured and implanted to provide stimulation therapy to one or more nerves and/or muscles that, for example, influence the behavior of musculature of the pelvic region of the patient, for example musculature relating to one or both of urinary incontinence and fecal incontinence (e.g., the external urethral sphincter 34, the internal urethral sphincter 32, pelvic floor muscles 18, the external anal sphincter, the internal anal sphincter, etc.). For example, stimulation can be provided to one or more of the pudendal nerve 44, the pelvic nerve 40, the sacral nerve, hypogastric nerve, or branches thereof. For example, stimulation can be provided to a deep branch of the pudendal nerve 44 or other nerve, for example applied to a distal-most branch of the pudendal nerve 44 (or other nerve) at or in highly close proximity to a location where the branch contacts or terminates a muscle (or other anatomical feature) of interest. With optional embodiments in which the treatment system 50 is configured and implanted to deliver stimulation to two (or more) target sites (e.g., two or more of the pudendal nerve 44, the pelvic nerve 50, the sacral nerve, the hypogastric nerve, etc., and/or two or more different locations along one incontinence amelioration-related nerve and/or different incontinence amelioration-related nerves, etc.), the so-applied simulation can be toggled (e.g., simultaneous, alternating, overlapping, unilateral, bilateral, selective), optionally while additionally toggling/adjusting one or more stimulation parameters e.g., amplitude, frequency, pulse width, duty cycle, pulse shape, etc.). Alternatively or in addition, the system 50 can apply electrical stimulation to tissue sites proximate a nerve or nerve branch of interest. In yet other embodiments, stimulation can be applied directly to a muscle. Various, non-limiting examples of stimulation protocols or algorithms are described in PCT Publication No. 2020/243104 (Rondoni, et al.) and PCT Publication No. WO 2022/192726 (Rondoni, et al.) the entire teachings of each of which are incorporated herein by reference.

With the above generalities in mind, the bioimpedance sensor(s) 62 is configured, located, and operated to sense bioimpedance (bioelectrical impedance) properties of the patient as described below, with the so-obtained bioimpedance information being informative to operations implemented by the control portion 70. As part of the bioimpedance arrangement, the bioimpedance sensor(s) 62 can serve to emit or receive electrical signals appropriate for generating and collecting relevant bioimpedance information. The impedance of human body tissue is able to provide information about the physiological and pathological properties of the tissue. In some embodiments, the bioimpedance sensors of the present disclosure operate by injecting electrical currents via a current injection or driving electrode and measuring the voltage (e.g., frequency dependent ac potential) generated at a voltage sensing electrode. In other embodiments, the bioimpedance sensors of the present disclosure consolidate the voltage sensing and current injection electrodes to a single electrode. In yet other embodiments, the bioimpedance sensors of the present disclosure incorporate the conductive housing of the IPG 64 as the current injection electrode and/or the voltage sensing electrode. Optionally, with these and related embodiments, the IPG 64 and the bioimpedance sensor 62 can be located at opposite sides of the patient's body; with these and other non-limiting examples, the impedance measurement vector may improve impedance sensitivity. In yet other embodiments, the stimulation element 66 can be used as the voltage sensing electrode of the bioimpedance sensor (for example during periods where stimulation is not being delivered) and/or as the current injection electrode (for example if the current injection pulses are interleaved with the stimulation pulses during a stimulation burst, the stimulation pulses are utilized for bioimpedance sensing). In yet other embodiments, two or more of these bioimpedance sensing formats can be utilized in a coordinated fashion. In the descriptions below, reference to a “bioimpedance sensor” can include any of these formats. With any of the embodiments of the present disclosure, impedance can optionally be measured during a therapeutic stimulation delivery pulse, and then again at a microamp level. Body tissue will demonstrate non-Ohmic behavior at low voltages, producing different values of impedance depending on what voltage is applied. The difference in impedance measured between different current and voltage levels may change as a function of tissue movement and may be used as an indication of an SUI causing event.

In some embodiments the bioimpedance sensor(s) 62 can be configured and located to facilitate the sensing of bioimpedance information implicating a condition of an anatomical structure of the patient, for example detecting pelvic floor motion that is otherwise indicative of increased pressure (or other circumstances associated with possible leakage or incontinence). Bioimpedance could be used to sense information indicative of one or more parameters of interest, such as bladder fullness, forces acting on the bladder indicative of a stress incontinence event or normal voiding, body motion, voiding events, leak events, potential leak triggers, etc. Bioimpedance can, in other examples, be used to assess bladder fullness or static pressure due to posture/activity. With these and related embodiments, this information can be used to adjust the sensitivity of an activity sensing algorithm programmed to, or operated by, the control portion 70; to adjust stimulation parameters including strength, frequency, amplitude, duty cycle, pulse shape, duration, stimulation location, turning stimulation on/off, etc., via stimulation delivery algorithm(s) programmed to, or operated by, the control portion 70; to monitor therapy effectiveness; optionally combined or evaluated with information provided by other sensors (e.g., movement sensor(s)) to determine when stimulation should be delivered; to inform the patient of a determined current condition of an anatomical structure (e.g., bladder fullness can be determined from bioimpedance sensor information and conveyed to the patient in response to a request by the patient and/or when the determined bladder fullness meets a designated criteria (e.g., exceeds a predetermined level), etc. In other embodiments, the bioimpedance information can be utilized in a similar manner for the treatment of bowel disorder(s), such as fecal incontinence. With any of the embodiments of the present disclosure, the bioimpedance sensor(s) 62 can be located as shown or can be located at other positions of the patient, for example at any location throughout the pelvis, abdomen, or back (anteriorly or posteriorly). The bioimpedance sensor(s) 62 can be electronically connected to or communicate with the control portion 70 in various fashions, including wired and wireless connections. Unless stated otherwise, any embodiment of the present disclosure showing or describing a wired connection can alternatively be configured for wireless communication.

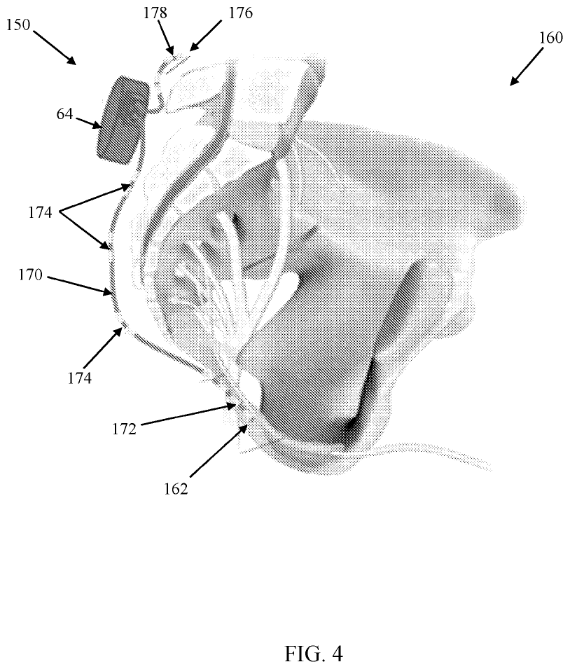

In some examples, bioimpedance can be used to measure a condition (e.g., movement, position, state, etc.) of the pelvic floor and/or related structures, generating information indicative of, for example, the onset of an SUI leak, event(s) known or deemed to cause an SUI leak, etc. With these and related embodiments, a multiple combination of current injection and voltage sensing electrodes can be employed to determine calculated values of impedance as part of a treatment system, such as a treatment system 150 shown in FIG. 4 as implanted within a patient 160. The treatment system 150 can, in many respects, be similar to other treatment systems of the present disclosure, for example the treatment system 50 of FIG. 3. In general terms, the treatment system 150 includes the IPG 64 implanted within the patient 160. A first lead 170 is connected to the IPG 64 and carries one or more stimulation elements (e.g., electrodes) 172. The first lead 170 extends from the IPG 64 to position the stimulation element(s) 172 at a desired stimulation target site (in the non-limiting example of FIG. 4, the stimulation element(s) 172 are positioned to deliver stimulation energy to a pudendal nerve 162 of the patient 160). The IPG 64 is programmed (or is prompted) to deliver stimulation signals to the stimulation element(s) 172 via the lead 170 that in turn are applied to the target site. It will be understood that locations of the IPG 64 and the stimulation element(s) 172 reflected in FIG. 4 are examples only, and are in no way limiting.

In addition to the stimulation element(s) 172, the first lead 170 carries one or more bioimpedance sensors 174. The bioimpedance sensors 174 can assume any of the formats described above, and in some embodiments each include at least one current injection electrode and at least one voltage sensing electrode. In the example of FIG. 4, each of the bioimpedance sensors 174 includes a current injection electrode/voltage sensing electrode pair, although other configurations are also acceptable (e.g., one or more or all of the bioimpedance sensors 174 can include two current injection electrodes and two voltage sensing electrodes; current injection and voltage sensing electrodes are consolidated into a single electrode; the conductive housing of the IPG 64 can serve as one of the current injection or voltage sensing electrodes; the stimulation element(s) 172 can serve as one of the current injection or voltage sensing electrodes). Further, while the example of FIG. 4 reflects three of the bioimpedance sensors 174 along a length of the first lead 170, any other number, either greater or lesser, is acceptable. Regardless, the IPG 64 is programmed (or is prompted) to operate each of the bioimpedance sensors 174 in a manner appropriate for determining bioelectrical impedance at tissue proximate the corresponding sensor 174 as is known in the art (e.g., operating the current injection electrode(s) to deliver, or inject, a current and the voltage sensing electrode(s) to measure voltage). Bioimpedance at each sensor 174 can be determined from the injection current and sensed parameters using conventional techniques as are known in the art. In some embodiments, providing one or more of the bioimpedance sensors 174 along a single lead that is connected to the IPG 64 can be effective in identifying bioimpedance changes indicative of events or conditions of interest, such as SUI leakage events.

The treatment system 150 optionally includes a second lead 176 connected to and routed from the IPG 64. The second lead 176 can assume a wide variety of forms, and can be provided with various features formatted for one or more functions of interest. For example, the second lead 176 can include one or more electrodes that are operated by the IPG 64 to perform one or more of stimulating, sensing, etc. In some embodiments, the second lead 176 can include one or more bioimpedance sensors 178. The bioimpedance sensors 178 can be akin to the bioimpedance sensors 174 as described above (e.g., each including at least one current injection electrode and at least one voltage sensing electrode; current injection and voltage sensing electrodes are consolidated into a single electrode; the conductive housing of the IPG 64 can serve as one of the current injection or voltage sensing electrodes; a stimulation element of the second lead 176 can serve as one of the current injection or voltage sensing electrodes), and are operated by the IPG 64 in a manner appropriate for determining bioelectrical impedance at tissue proximate the corresponding sensor 178 as is known in the art.

In other embodiments, additional leads can be provided that each carry one or more bioimpedance sensors as described above. With these and related embodiments, the two or more leads, each with bioimpedance sensor(s), can be more effective than a single lead in evaluating three dimensional changes in bioimpedance changes indicative of events or conditions of interest, such as SUI leakage events, event(s) known or deemed to cause an SUI leak, etc., providing the system 150 with adaptive bioimpedance sensing features. Adaptive bioimpedance methods of the present disclosure can be based on feedback from the patient or other detectable means. The control portion 70 (FIG. 3) of the system 150 can be programed to automatically select the most effective type of bioimpedance sensing (e.g., which of the multiple bioimpedance sensors is generating the most relevant indication of an actual or potential leak event at a given point in time) correlating to an actual leak event. Alternatively or in addition, the treatment system 150 can include features appropriate for the measurement of impedances in two or more vectors (e.g., perpendicular vectors), for example two or more vectors across an abdomen of the patient, such that the ratio of the impedances can produce greater sensitivity than either vector alone since one will potentially increase in measured impedance while the perpendicular vector may decrease in impedance when an SUI lead-causing event occurs.

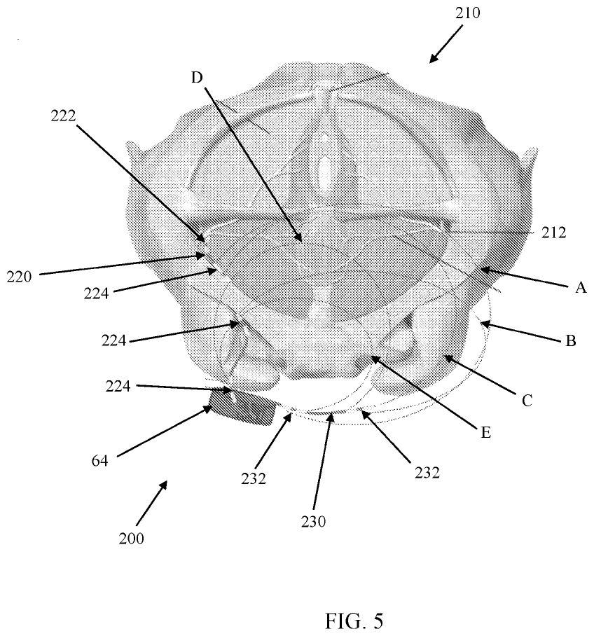

In some embodiments, the treatment systems of the present disclosure can be configured and programmed to obtain bioimpedance measurements at multiple points and in a predetermined pattern (e.g., sequential). For example, FIG. 5 illustrates another treatment system 200 in accordance with principles of the present disclosure as implanted to a patient 210. The treatment system 200 can, in many respects, be similar to other treatment systems of the present disclosure, for example the treatment system 50 of FIG. 3. In general terms, the treatment system 200 includes the IPG 64 implanted within the patient 210. A first lead 220 is connected to the IPG 64 and carries one or more simulation elements (e.g., electrodes) 222. The first lead 220 is routed from the IPG 64 to position the stimulation element(s) 222 at a desired stimulation target site (in the non-limiting example of FIG. 5, the stimulation element(s) 222 are positioned to deliver stimulation energy to a pudendal nerve 212 of the patient 210). The IPG 64 is programmed (or is prompted) to deliver stimulation signals to the stimulation element(s) 222 via the lead 220 that in turn are applied to the target site. It will be understood that locations of the IPG 64 and the stimulation element(s) 222 reflected in FIG. 5 are examples only, and are in no way limiting.

In some embodiments, the first lead 220 additionally carries one or more bioimpedance sensors 224. The bioimpedance sensors 224 can be akin to the bioimpedance sensors as described above (e.g., each including at least one current injection electrode and at least one voltage sensing electrode; current injection and voltage sensing electrodes are consolidated into a single electrode; the conductive housing of the IPG 64 can serve as one of the current injection or voltage sensing electrodes; the stimulation element(s) 222 can serve as one of the current injection or voltage sensing electrodes), and are operated by the IPG 64 in a manner appropriate for determining bioelectrical impedance at tissue proximate the corresponding sensor 224 as is known in the art.

The system 200 can further include a second lead 230 connected to and routed from the IPG 64. The second lead 230 can assume a wide variety of forms, and can be provided with various features formatted for one or more functions of interest. For example, the second lead 230 can include one or more electrodes that are operated by the IPG 64 to perform one or more of stimulating, sensing, etc. In some embodiments, the second lead 230 can include one or more bioimpedance sensors 232. The bioimpedance sensors 232 can be akin to the bioimpedance sensors as described above (e.g., each including at least one current injection electrode and at least one voltage sensing electrode; current injection and voltage sensing electrodes are consolidated into a single electrode; the conductive housing of the IPG 64 can serve as one of the current injection or voltage sensing electrodes; the stimulation element(s) (where provided) can serve as one of the current injection or voltage sensing electrodes), and are operated by the IPG 64 in a manner appropriate for determining bioelectrical impedance at tissue proximate the corresponding sensor 232 as is known in the art.

The IPG 64 can be programmed, or can be prompted, to obtain bioimpedance measurements via the bioimpedance sensors 224, 232 in a sequential pattern. Several possible impedance sensing vectors available with the bioimpedance sensors 224, 232 are labeled at A-E in FIG. 5. The bioimpedance measurements at multiple points and in a sequential pattern can provide a more complete three dimensional characterization of anatomy of interest (e.g., the pelvic floor), facilitating reliable or predictive evaluation of bioimpedance changes that can be determined to be indicative of events or conditions of interest, such as SUI leakage events. In some embodiments, the bioimpedance sensors can be arranged to sense impedance in two or more vectors (e.g., perpendicular vectors), generating a perpendicular vector ratio or other metric as described above. The ratio of impedance and changing ratio between vectors can be considered as an indication of an SUI causing event in some embodiments.

As a point of reference, while FIG. 5 depicts the IPG 64 placed opposite the spine from the stimulation location, other arrangements are acceptable. For example, the IPG 64 can be placed on the same side of the spine, as well as on the abdomen of the patient. This abdominal placement may be particularly appropriate for situations in which the lead 220 is anteriorly placed.

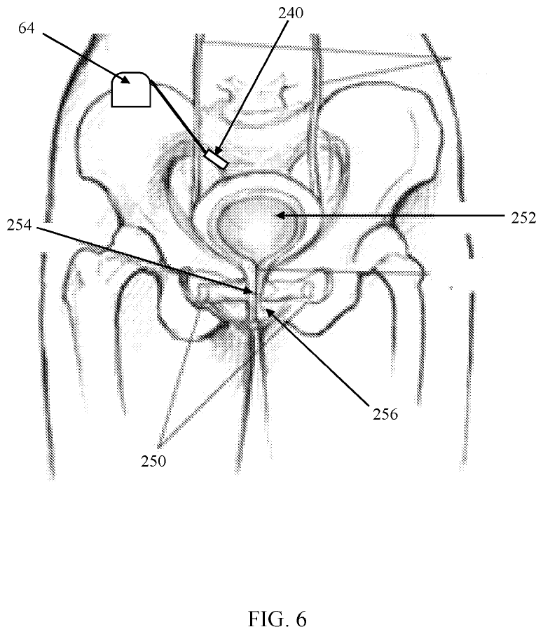

In addition, or as an alternative, to obtaining bioimpedance information that is useful to inform or predict onset or occurrence of an event or condition of interest (e.g., SUI leakage), some systems and methods of the present disclosure obtain and/or utilize bioimpedance information to monitor a condition (e.g., movement, position, state, etc.) of anatomical structures understood to be favorable for preventing a SUI leakage events. For example, and with reference to FIG. 6, systems of the present disclosure can include the IPG 64 and one or more bioimpedance sensors 240 (schematically represented in FIG. 6) as described above that is located and configured to detect movement (e.g., a desired movement, an undesired movement, etc.) of one or more of the pelvic floor 250, bladder 252, urethra 254, vagina 256 (referenced generally) and/or other anatomical structures deemed as being favorable for preventing SUI leakage events. Alternatively or in addition, the bioimpedance sensors 240 can be located and configured to detect other condition(s) of interest likely relevant to continence, such as position or state.

The IPG 64 can be programmed, or can be prompted (via the control portion 70 (FIG. 3)), to consider or review the bioimpedance information of the anatomical structure being monitored in performing or implementing various actions. For example, with some systems and methods of the present disclosure, the IPG 64 can be programmed, or can be prompted via the control portion 70, to deliver functional stimulation to muscles of the pelvic floor (e.g., by an appropriately placed lead electrode connected to the IPG 64) so as to facilitate the desired movement of the anatomical structure(s) being monitored. In some examples, functional stimulation can be delivered coincident with increases in exertion and/or intraabdominal pressure as measured through changes in bioimpedance.

Alternatively or in addition, bioimpedance-based monitoring of the condition (e.g., movement, position, state, etc.) of one or more anatomical structures deemed as being favorable for preventing SUI leakage events can, in some examples, serve as feedback information from which the level of functional stimulation delivered by the IPG 64 to pelvic floor muscle(s) is modulated so as to maintain the pelvic floor structures in a position favorable to continence. For example, a desired position of the anatomical structure being monitored corresponding with likely continence by the patient can be predetermined or can be learned over time; when functionally stimulating the pelvic floor muscle(s) for purposes of increasing likelihood of continence, the IPG 64 can be programmed, or prompted (via the control portion 70 (FIG. 3)), to implement a functional stimulation protocol that results in the monitored anatomical structure being arranged in the desired position. In yet other examples, the movement (or other condition) of the anatomical structure such as the bladder and/or the urethra are detectable, and thus can be monitored, using the bioimpedance sensor(s) 240 and can be affected by functional stimulation; monitoring and effectively “correcting” the anatomical structure to the desired position (or other condition) is akin or similar to a suspension procedure known to treat SUI. In this way, a closed loop control of tissue position can be maintained. With these and related embodiments, an optimal implant location of the bioimpedance sensor(s) 240 and/or other components of the treatment system can be identified by external skin sensors (not shown) or the like.

In yet other examples, systems and methods of the present disclosure can include utilizing bioimpedance sensor(s) 240 to detect a condition (e.g., movement, position, state, etc.) of the pelvic floor 250 in ways that are beneficial or favorable to continence, and provide a signal as feedback to the patient that a condition (e.g., movement/state) has been achieved, for example by applying a stimulation signal that is perceptible to the patent. For example, a particular movement of the pelvic floor 250 corresponding with likely continence by the patient can be predetermined or can be learned over time; the particular movement could be identified in response to an input to the control portion 70 (FIG. 3) by the patient when activities exist without leakage, when a marker is input by the patient when leaks are present, etc. Regardless, the IPG 64 can be programmed, or prompted, to deliver a feedback signal to the patient (e.g., a brief series of perceptible pulses delivered by the IPG 64 to one or more stimulation element (not shown)) when the bioimpedance information implicates the patient has achieved, or is achieving, the desired particular movement, position, state, etc. With these and related techniques, the patient is thus made aware that s/he is accomplishing a desired pelvic floor movement (or other condition). Thus, for example, during a training session (e.g., a session that the patient can start and stop and during which the IPG 64 operates to provide the feedback signals), a patient can practice or attempt to achieve the desired pelvic floor movement and is directly made aware of successful performance. Alternatively or in addition, where the bioimpedance information implicates that the monitored condition (e.g., movement, position, state, etc.) is undesired, a feedback signal can be delivered to the patient.

In yet other examples, the bioimpedance sensor(s) 240 can be operated to detect a condition (e.g., movement, position, state, etc.) of the pelvic floor 250 in response to or in association with delivered stimulation. With these and related embodiments, the IPG 64 can be programmed, or prompted (via the control portion 70 (FIG. 3)) to automatically titrate the stimulation being delivered (e.g., one or more of strength, frequency, duty cycle, duration, etc.) to achieve the desired results. This auto-titration arrangement can further include, or consider, patient/clinician input, such as parameter ranges and comfort levels.

While FIG. 6 generally reflects the bioimpedance sensor 240 as being positioned external the anatomical structure of interest, other locations are envisioned. For example, in some embodiments the bioimpedance sensor 240 (e.g., the current source and measurement electrodes) can be placed within the bladder 252. This arrangement can facilitate isolating the effects of bladder volume on the measured impedance, for example to provide a more robust representation or estimation of the volume of fluid in the bladder 252. With these and related embodiments, the IPG 64 can be programmed, or can be prompted (via the control portion 70 (FIG. 3)), to implement or adjust a designated stimulation therapy regimen based upon a determined fullness of the bladder 252 (as implicated by information of the bioimpedance sensor 240). In other embodiments, a bioimpedance sensor arrangement can be provided by an electrode or the like positioned in the bladder 252 and operated as the active current source. For example, the active current source electrode can be operated to deliver tonic or intermittent current wave forms that can be detected or sensed by other electrode or electrode-like components (e.g., carried by a lead that is connected to the IPG 64) located outside the bladder 252 that thus function as the voltage sensing component. In addition to being employed for determining bioimpedance, the voltage sending electrode(s) can also be utilized to sense other physiologically relevant signals (EMG of pelvic muscles, abdominal muscles, back muscles, electroneurography (ENG), etc.). Regardless, the optional arrangement (with an active current source electrode located inside the bladder) can be useful for detecting or implicating relative motion of pelvic and/or abdominal tissues, detecting information useful in determining or estimating a volume of urine in the bladder 252, etc.

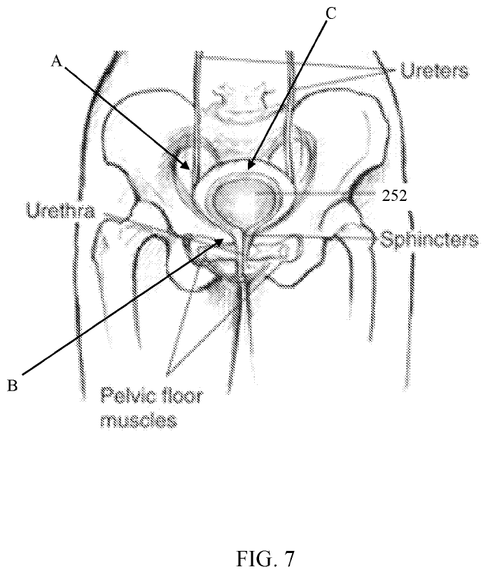

Other techniques or arrangements for obtaining bioimpedance information relating to the bladder 252 can be employed. With reference to FIG. 7, the IPG 64 (not illustrated in FIG. 7 for ease of understanding) and the stimulation element(s) are, in some applications, implanted on the same side of the patient's body (e.g., in FIG. 7, a possible implant location of the IPG 64 is indicated generally at A, and a possible implant location of the stimulation element is indicated generally at B). With these and similar implant locations, the impedance vector between the IPG 64 and the stimulation element may cross only part of the bladder, and can be sensitive enough to detect direct changes in volume as well as indirect changes related to the relative positioning of the IPG 64 and the stimulation element(s) as well as other tissues as the bladder volume changes. In related embodiments in which the stimulation element is implanted at a target site along, for example, the bladder 252, the bioimpedance sensor 62 (not shown) can be provided as part of a sensor lead body electrically coupled to the IPG 64 assembly and arranged to locate the bioimpedance sensor 62 along the bladder 252 generally opposite the stimulation element. In yet other embodiments, a bioimpedance signal delivery element (e.g., an electrode) can be provided apart from the stimulation element and that is implanted at a location generally away from a location of the bioimpedance sensor 62. For example, a second impedance sensing lead (carrying one, two or more electrodes) can be inserted from the pocket at which the IPG 64 is implanted to a location (e.g., indicated generally at C in FIG. 7) to improve the measurement vector for impedance and/or allowing the selection of a possible impedance vector between the stimulation element, the electrode(s) of the impedance sensing lead, and the IPG 64. In yet other embodiments, the bioimpedance sensor 62 can be configured to be externally worn by the patient, and provided as part of a sensor unit that delivers sensed information wirelessly to the control portion 70.

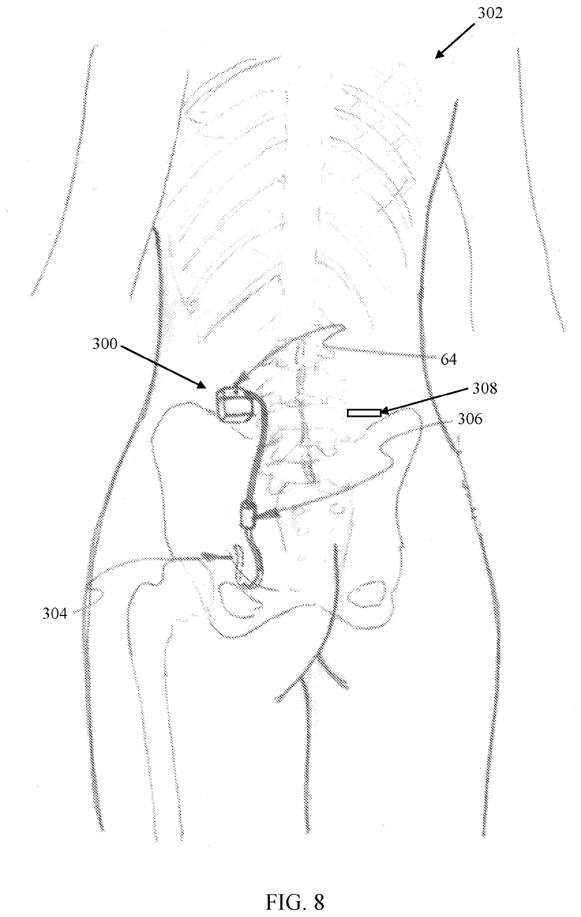

In addition, or as an alternative, to active bioimpedance-based monitoring of targeted tissue (e.g., anatomical structures) of the patient, some systems and methods of the present disclosure can include a passive electrical element separately installed or implanted to the patient at a location intended to affect the electric field being sensed by the bioimpedance sensor in a manner that can be correlated with events or conditions of interest. For example, FIG. 8 illustrates another system 300 in accordance with principles of the present disclosure implanted to a patient 302. The system 300 includes the IPG 64 and a stimulation lead 304 routed from the IPG 64 to position one or more stimulation elements (e.g., electrode(s)) at a desired stimulation target site (e.g., the pudendal nerve, deep branch of the pudendal nerve or other nerve, etc.). A bioimpedance sensor 306 is connected to the IPG 64, and can assume any of the formats described above. While the bioimpedance sensor 306 is illustrated as being carried by the stimulation lead 304, in other embodiments, the bioimpedance sensor 306 (or components thereof) can be connected to the IPG 64 apart from the stimulation lead 304. Regardless, the current emitting electrode(s) and voltage sensing electrode(s) of the bioimpedance sensor 306 can be located on one side of the body convenient to lead placement (e.g., lumbar back subcutaneous tissue).

The system 300 additionally includes a passive electrical element 308. The passive electrical element 308 is separate or apart from the bioimpedance sensor 306 (and any structure, such as a lead body, carrying the bioimpedance sensor 306), and can assume various forms capable of affecting an electric field and resulting impedance signature of the bioimpedance sensor 306. For example, the passive electrical element 308 can be a separate lead, a metallic structure, etc. Regardless of exact form, the passive electrical element 308 is implanted at a location away from the bioimpedance sensor 306 (e.g., ventral abdominal subcutaneous tissue). With this arrangement, as the passive electrical element 308 moves relative to the electrodes of the bioimpedance sensor 306, the electric field and resulting impedance signature will change. In some embodiments, by locating the bioimpedance sensor 306 and the passive electrical element 308 at opposite sides of the body, the impedance measurement vector may improve impedance sensitivity. In some embodiments, the IPG 64 can be programmed, or can be prompted, to control delivery of functional stimulation energy (e.g., of the pudendal nerve) based on the impedance signature that is designated (e.g., predetermined or learned over time) as correlating with an SUI leak causing event.

The passive electrical element 308 can be implanted or positioned at a various locations other than the location implicated by FIG. 8. In some embodiments, a location of the passive electrical element 308 can be selected to more directly implicate a condition (e.g., movement, position., state, etc.) of an anatomical structure understood to directly implicate the bladder and/or bowel dysfunction being treated by the system 300. For example, the passive electrical element 308 can be implanted on the pelvic floor, such as to a muscle of the pelvic floor such as the levator ani or transverse perineum. In other embodiments, the passive electrical element 308 is placed in or on the bladder. For example, the passive electrical element 308 can be delivered to the bladder using minimally invasive surgery such as delivering the passive electrical element 308 through a cystoscope and anchoring to the bladder wall with a suture, clip, etc. Other techniques for delivering the passive electrical element 308 to (or in) the bladder are also acceptable. Moreover, the passive electrical element 308 can be held in a more central location away from the bladder walls through deployable fibers that will easily collapse when the passive electrical element 308 needs to be extracted back through the urethra.

Returning to FIGS. 1-3, in other embodiments the bioimpedance sensor(s) 62 can be located or positioned within the patient at a variety of other locations to provide information useful for bladder and/or bowel dysfunction treatment. With embodiments in which the bioimpedance sensor 62 is formatted to use the same electrode to pass the current and measure the voltage needed to pass that current (such that only two electrodes are needed to measure the impedance), the return electrode can be placed nearer or further from the current source electrode in order to measure impedances from smaller or larger volumes of tissue or fluid. With these and related embodiments, an arrangement of the bioimpedance sensor components relative to the patient can be selected in accordance with the targeted tissue or fluid to be monitored. For example, where the targeted tissue/structure is relatively large, the return electrode can be placed further away as compared to a return electrode location for a relatively small, targeted tissue/structure. Moreover, a location of the return electrode can be effective to measure changing impedances locally, across the structures experiencing the greatest change in bioimpedance and not including the volume of other structures not changing impedance so as to not dilute the signal.

In some optional embodiments, and with any of the bioimpedance sensor arrangements of the present disclosure, the bioimpedance sensor 62 can be configured or operated to detect or obtain information in addition to bioimpedance by detecting the impedance signal simultaneously with other signals via means of separating the frequencies of the measured signals. For example, the current waveforms emanating from the injection source electrode(s) of the bioimpedance sensor 62 can be sinusoidal with a frequency outside frequency band of other signals of interest, such as electromyography (EMG), electroneurography (ENG), etc. Filters in the electronics of the bioimpedance sensor 62 and/or the IPG 64 can separate out the other signals of interest (e.g., signals due to EMG or ENG) from the bioimpedance-related signals (i.e., due to the impedance current source(s)). In other, related embodiments, the bioimpedance sensor 62 can be configured or operated to generate discrete pulses. The discrete pulses can be charge balanced but can be delivered at a variety of frequencies and/or waveforms (e.g., square, exponential, saw tooth, etc.). This approach (i.e., discrete pulses) can be more energy efficient (as compared to a more continuous signal) thus leading to enhanced battery life. In yet other examples, the discrete pulses can provide a different (broader spectrum), complex impedance signature that can be used to give a better overall perspective of the tissue/movement of interest. Alternatively or in addition, a frequency sweep can be utilized to achieve similar results. Likewise, one or more discrete frequencies can be employed.

In other embodiments, the bioimpedance sensor format can include positioning or locating two or more current injection sources (e.g., electrodes) at various/different locations. During use, the injection sources are operated (e.g., via programming of the IPG 64 and/or the control portion 70) to produce signals of various/different frequencies. The corresponding voltage sensing component/detection circuitry measures impedance relative to each of the different frequencies, allowing the detection circuitry to measure multiple impedances simultaneously and disambiguated from each other. Some examples implement waveform morphology analysis, such as a decaying exponential current injection pulse being analyzed to determine changes in morphology over time that in turn can indicate or implicate an SUI causing event for the patient. In related embodiments, by locating the two (or more) current source electrodes at different distances from the voltage sensing component, the systems and methods of the present disclosure can preferentially measure bioimpedance nearer or further from the sources, even if the voltage sensing component/electrodes are in the same location. In some instances, local impedance variations can confound the desired bulk impedance changes when the voltage is measured from the same electrodes as the current injection is done. By separating the sensing electrodes from the current electrodes, this potential concern can be minimized.

In some embodiments, the systems and methods of the present disclosure can be configured or programmed to leverage the understanding that impedance of tissues and fluid varies with the frequency at which it is measured, with some tissues varying more than others. With this in mind, some systems and methods of the present disclosure operate the bioimpedance sensor(s) to sweep through a set of frequencies and/or measure impedance at multiple frequencies. With these and related techniques, the IPG 64 can be programmed and/or prompted by the control portion 70 to differentiate the movement of solid tissue, such as muscles and organs, form changes in bulk fluid such as in the bladder. These and similar strategies can be used alone or in combination with one or more other bioimpedance sensor modalities and techniques of the present disclosure.

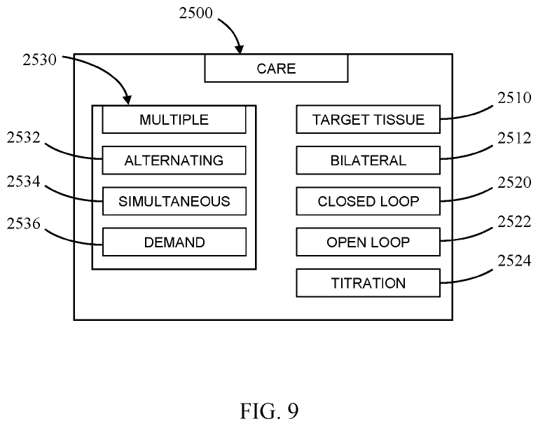

FIG. 9 is a block diagram schematically representing a care engine 2500 of a control portion. In some examples, the care engine 2500 may comprise an example implementation of, and/or at least some of substantially the same features and attributes as, any of the IPGs, care engines and/or the control portions (e.g., FIGS. 3-8) of the present disclosure. Accordingly, the various functions and parameters of the care engine 2500 may be implemented in a manner supportive of, and/or complementary with, the various functions, parameters, portions, etc., of any of the devices and control portions and/or various functions, parameters, portions, etc., relating to stimulation throughout examples of the present disclosure. In some examples, the care engine 2500 may include an implementation of the control portion 70 of FIG. 3.

In some examples, different target tissue may be stimulated using at least one stimulation element. The target tissues may be stimulated at the same time (e.g., simultaneously or overlapping times) or at different times and/or in response to different sensed parameters, such as those described and illustrated in connection with at least FIGS. 3-8.

In some examples, any of the methods, apparatuses, and/or devices may be used to provide bladder and/or bowel dysfunction care to different target tissue, including those described in connection with at least FIGS. 1-8.

As shown by FIG. 9, in some examples, via target tissue parameter 2510, stimulation may be delivered to select target tissue such as, but not limited to, the pudendal nerve, the pelvic nerve, the sacral nerve, hypogastric, or branches thereof. In some examples, target tissues may include any muscles which affect and/or promote continence (e.g., urethral sphincters, detrusor, etc.) and/or nerves which innervate such muscles. In some examples, target tissue includes a combination of nerves and/or muscles such as, but not limited to, terminal fiber ends of nerves where a nerve ending terminates into (or at) the muscle being innervated.

In some examples, in addition to or instead of selecting different tissue for stimulation, the target tissue parameter 2510 may comprise adjusting care parameters (e.g., stimulation parameters) via selecting between (or using a combination of) various locations along a nerve such as stimulating multiple different sites along a particular nerve.

In some examples, in addition to or instead of selecting different nerves for stimulation, the target tissue parameter 2510 may comprise adjusting care parameters via selecting between (or using a combination of) different fascicles within a particular nerve in order to selectively stimulate target efferent fibers while omitting (or minimally impacting) stimulation of other, non-target fibers and/or to selectively stimulate target efferent fibers while omitting (or minimally impacting) stimulation of other, non-target fibers.

In some examples, the care engine 2500 may implement stimulation according to a bilateral parameter 2512 in which stimulation is applied to target tissue on both sides (e.g., left and right) of the patient's body. In some such examples, the bilateral stimulation may be delivered to the same target tissue (e.g., pudendal nerve, pelvic nerve, sacral nerve, hypogastric, or branches thereof) on both sides of the body. However, in some examples, the bilateral stimulation may be delivered to different target tissue or tissue on a left side of the body while stimulating another nerve or tissue on a right side of the body, or vice-versa.

In some examples, the bilateral parameter 2512 may be implemented in a manner complementary with the alternating parameter 2532, simultaneous parameter 2534, or demand parameter 2536 of multiple function 2530, as further described below.

In some examples, the care engine 2500 may comprise a multiple function 2530 by which various care parameters may be implemented in dynamic arrangements. In some such examples, the care engine 2500 may comprise an alternating parameter 2532 by which care provided to one target tissue (e.g., pudendal nerve) may be alternated with care provided to at least one other target tissue (e.g., pelvic nerve). However, the alternating parameter 2532 also may be applied in combination with the bilateral parameter 2512 to apply care to the target tissue (or different target tissue) on opposite sides of the body in which care may be applied on a left side of the body and then applied on the right side of the body in an alternating manner. As used herein, applying or providing care to target tissue may include applying stimulation and/or mechanically maneuvering the target tissue.

In some examples, the care engine 2500 may comprise a simultaneous parameter 2534 by which care may be applied simultaneously to at least two different target tissues. In some examples, the at least two different target tissues comprise two different tissues, such as the pudendal nerve and the pelvic nerve. In some examples, the at least two different target tissues may comprise two different locations along the same tissue or two different fascicles of the same nerve. In some examples, the simultaneous parameter 2534 may apply stimulation per bilateral parameter 2512 simultaneously on opposite sides of the body to the same tissue or different tissue, and/or apply mechanical maneuvering simultaneously on opposite sides of the body to the same tissue.

In some examples, the care engine 2500 may comprise a demand parameter 2536 by which care may be applied to at least one target tissue on a demand basis. For example, stimulation may be applied to one nerve (e.g., pudendal nerve, such as a deep perineal branch thereof) which may be sufficient to achieve the patient metric (e.g., continence) for most circumstances, but may become insufficient for some situations. In the latter situation, to achieve the target patient metric, via the demand parameter 2536, stimulation of a different nerve (e.g., pelvic nerve) may be implemented in addition to, or instead of, stimulation of the first nerve (e.g., pudendal nerve) which was previously being stimulated. In some examples, the first or primary nerve being stimulated may be a nerve other than the pudendal nerve.

In some examples, the care engine 2500 also may further implement at least some aspects of the control portion of FIGS. 3-8 and/or according to at least one of a closed loop parameter 2520, open loop parameter 2522, and nightly titration parameter 2524.

In some examples, the care engine 2500 comprises a closed loop parameter 2520 to deliver care based on sensed patient physiologic information and/or other information (e.g., environmental, temporal, captured by an external system and communicated to the care engine 2500, etc.). In some such examples, via the closed loop parameter 2520 the sensed information may be used to control the particular timing of the care according to bladder fullness information. In some such examples and as previously described, the bladder fullness information and/or other information used with the closed loop parameter 2520 may be determined via the sensors, devices, sensing portions, as previously described in association with at least FIGS. 3-8.

In some examples, the care engine 2500 comprises an open loop parameter (e.g., 2522 in FIG. 9) by which bladder and/or bowel dysfunction care (e.g., “use”) is applied without a feedback loop of sensed physiologic information. In some such examples, in an open loop mode the care is applied during a treatment period without (e.g., independent of) information sensed regarding the patient's bladder fullness, detrusor levels, etc.

In some examples, the care engine 2500 comprises a titration parameter 2524 by which an intensity of the bladder and/or bowel dysfunction therapy may be titrated (e.g., adjusted) to be more intense (e.g., higher stimulation amplitude, greater frequency, and/or greater pulse width) or to be less intense within a treatment period.

In some such examples, the titration parameter 2524 may be implemented according to at least some aspects of the example methods and/or example devices of FIGS. 3-8. Accordingly, in some examples, the titration parameter may be implemented as automatic titration, while in some examples, the titration parameter may be implemented via manual titration by a patient (or clinician), such as to adjust one or more stimulation parameters. In some examples, the titration parameter may be implemented via combination of patient/manual titration and automatic titration to guide the patient in a manner complementary with manual titration.

In some examples, at least some aspects of the titration parameter 2524 of the care engine 2500 and/or at least some aspects of titration as generally disclosed throughout FIGS. 3-8 in examples of the present disclosure may comprise (and/or may be implemented) in a manner complementary with and/or via at least some of substantially the same features and attributes as described in: (i) PCT Publication No. 2020/243104 (Rondoni, et al.), and (ii) PCT Publication No. WO 2022/192726 (Rondoni, et al.), each of which are hereby incorporated by reference in their entirety.

The various ranges provided herein include the stated range and any value or sub-range within the stated range. Furthermore, when “about” is utilized to describe a value, this includes, refers to, and/or encompasses variations (up to +/−10%) from the stated value.

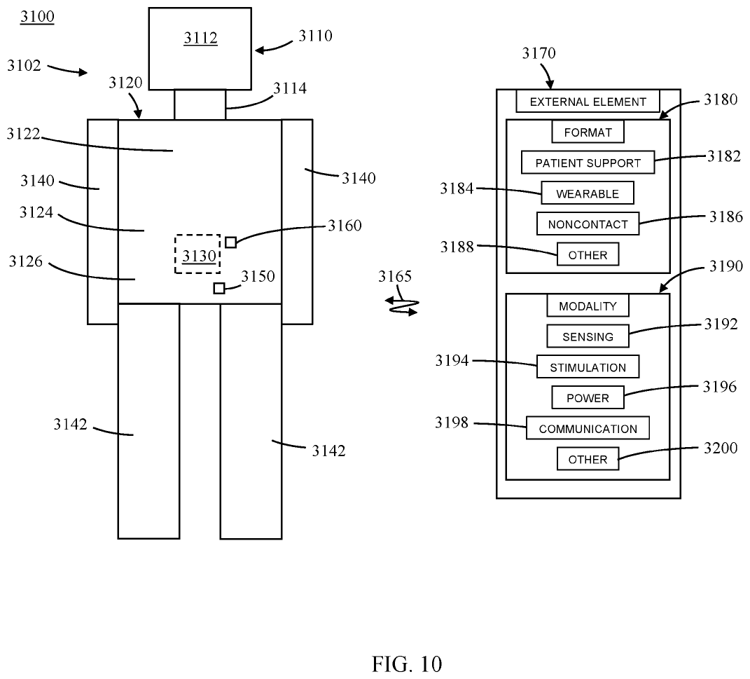

FIG. 10 is a block diagram schematically representing an example arrangement 3100 including patient's body 3102, including example target portions 3110-3142 at which at least some example sensing element(s) and/or stimulation elements may be employed to implement at least some examples of the present disclosure.

As shown in FIG. 10, patient's body 3102 comprises a head-and-neck portion 3110, including head 3112 and neck 3114. As further shown in FIG. 10, the patient's body 3102 comprises a torso 3120, which comprises various organs, muscles, nerves, other tissues, such as but not limited to those in pectoral region 3122 (e.g., lungs, cardiac), abdomen 3124, and/or pelvic region 3126. Organs, muscles, nerves, other tissues of the abdomen 3124 and/or the pelvic region 3126 include bladder 3130, urethra, anus, pelvic floor, etc. As further shown in FIG. 13, the patient's body 3102 comprises limbs such as arms 3140 and legs 3142.

It will be understood that various sensing elements (and/or stimulation elements) as described throughout the various examples of the present disclosure may be deployed within the various regions of the patient's body 3102 to sense and/or otherwise diagnose, monitor, treat various physiologic conditions such as, but not limited to the above-described examples in association with FIGS. 3-9. In some such examples, a stimulation element 3150 may be located in or near the pelvic region 3126 for treating bladder and/or bowel dysfunction (and/or near other nerves/muscles at the same or different location to treat bladder and/or bowel dysfunction and/or other conditions) and/or a sensing element 3160 may be located anywhere within the torso 3120 (or other body regions) to sense physiologic information for providing patient care.

In some examples, at least a portion of the stimulation element 3150 may comprise part of an implantable component/device, such as an IPG whether full sized or sized as a microstimulator. The implantable components (e.g., IPG, other) may comprise a stimulation/control circuit, a power supply (e.g., non-rechargeable, rechargeable), communication elements, and/or other components. In some examples, the stimulation element 3150 also may comprise a stimulation electrode and/or stimulation lead connected to the implantable pulse generator.

Further details regarding a location, structure, operation and/or use of the sensing element 3160, external element(s) 3170, and/or stimulation element 3150 are described above in association with at least FIGS. 3-9.

In some examples, any one of the implantable systems or apparatuses (or a combination thereof) may be implemented as part of the example arrangement 3100 of FIG. 10 instead of, or in addition to (e.g., in complementary relation to), the stimulation element 3150, with at least some examples throughout the disclosure providing further details of such example arrangements. Moreover, at least some aspects (e.g., sensing, control, etc.) associated with an implantable system or apparatus as described in association with FIGS. 3-9 also may be implemented, in whole or part, via external element 3170 of FIG. 10.

In some examples, at least a portion of the stimulation element 3150 may comprise part of an external component/device such as, but not limited to, the external component comprising a pulse generator (e.g., stimulation/control circuitry), power supply (e.g., rechargeable, non-rechargeable), and/other components. In some examples, a portion of the stimulation element 3150 may be implantable and a portion of the stimulation element 3150 may be external to the patient.

Accordingly, as further shown in FIG. 10, the various sensing element(s) 3160 and/or stimulation element(s) 3150 implanted in the patient's body may be in wireless communication (e.g., connection 3165) with at least one external element 3170.

As further shown in FIG. 10, in some examples, the external element(s) 3170 may be implemented via a wide variety of formats such as, but not limited to, at least one of the formats 3180 including a patient support 3182 (e.g., bed, chair, sleep mat, other), wearable elements 3184 (e.g., finger, wrist, head, neck, shirt), noncontact elements 3186 (e.g., watch, camera, mobile device, other), and/or other elements 3188.

As further shown in FIG. 10, in some examples, the external element(s) 3170 may comprise one or more different modalities 3190 such as (but not limited to) a sensing portion 3192, stimulation portion 3194, power portion 3196, communication portion 3198, and/or other portion 3200. The different portions 3192, 3194, 3196, 3198, 3200 may be combined into a single physical structure (e.g., package, arrangement, assembly), may be implemented in multiple different physical structures, and/or with just some of the different portions 3192, 3194, 3196, 3198, 3200 combined together in a single physical structure.

Among other such details, in some examples the external sensing portion 3192 and/or implanted sensing element 3160 may comprise an example implementation of, and/or at least some of substantially the same features and attributes as, the examples further described above in association with FIGS. 3-9.

In some examples, the external stimulation portion 3194 and/or implanted stimulation element 3150 may comprise at least some of substantially the same features and attributes of at least the stimulation arrangements, as further described above in association with at least FIGS. 3-9 and/or other examples throughout the present disclosure.

In some examples, the external power portion 3196 and/or power components associated with implanted stimulation element 3150 may comprise at least some of substantially the same features and attributes of at least the stimulation arrangements, as further described in association with at least FIGS. 3-9 and/or other examples throughout the present disclosure. In some such examples, the respective power portion, components, etc. may comprise a rechargeable power element (e.g., supply, battery, circuitry elements) and/or non-rechargeable power elements (e.g., battery). In some examples, the external power portion 3196 may comprise a power source by which a power component of the implanted stimulation element 3150 may be recharged.

In some examples, the wireless communication portion 3198 (e.g., connection/link at 3165) may be implemented via various forms of radiofrequency communication and/or other forms of wireless communication, such as (but not limited to) magnetic induction telemetry, Bluetooth (BT), Bluetooth Low Energy (BLE), near infrared (NIF), near-field protocols, Wi-Fi, Ultra-Wideband (UWB), and/or other short range or long range wireless communication protocols suitable for use in communicating between implanted components and external components in a medical device environment.

Examples are not so limited as expressed by other portion 3200 via which other aspects of implementing medical care may be embodied in external element(s) 3170 to relate to the various implanted and/or external components described above.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein.

1.-46. (canceled)

47. A method of treating a bladder and/or bowel dysfunction of a patient, the method comprising:
sensing at least one bioimpedance parameter of the patient; and
applying stimulation energy to an anatomical structure of the patient as a function of the sensed bioimpedance parameter.

48. The method of claim 47, wherein the step of sensing includes reviewing information generated by a bioimpedance sensor implanted within the patient.

49. The method of claim 48, wherein the bioimpedance sensor includes a current injection component and a voltage sensing component consolidated to a single electrode.

50. The method of claim 48, wherein in the step of applying stimulation energy includes operating an implantable pulse generator to deliver the stimulation energy, and further wherein a conductive housing of the implantable pulse generator is operated as one of a current injection component and a voltage sensing component of the bioimpedance sensor.

51. The method of claim 47, wherein the step of sensing includes measuring, via a bioimpedance sensor arrangement, movement of a pelvic floor structure of the patient indicative of an onset of at least one of a dysfunction condition and event.

52. The method of claim 51, wherein the bioimpedance sensor arrangement is carried by a single lead and includes one of:
a single current injection electrode and a plurality of voltage sensing electrodes;
a plurality of current injection electrodes and a single voltage sensing electrode; and
a plurality of current injection electrodes and a plurality of voltage sensing electrodes.

53. The method of claim 47, wherein the step of sensing includes:
monitoring, via a bioimpedance sensor arrangement, a condition of at least one of a pelvic floor, bladder, urethra, vagina and other structure of the patient favorable to continence; and
assessing whether the monitored condition corresponds with a designated condition movement.

54. The method of claim 53, wherein the condition is at least one of movement, position, and state.

55. The method of claim 53, wherein the condition if one of a desired condition and an undesired condition.

56. The method of claim 53, further comprising:
delivering functional stimulation to initiate a change in the pelvic floor when the monitored condition is determined to not correspond with the designated condition.

57. The method of claim 53, wherein the structure being monitored is one of the bladder and the urethra, the method further comprising:
delivering functional stimulation to initiate a change in the monitored structure when the monitored condition is determined to not correspond with the designated condition.

58. The method of claim 57, wherein the monitored condition is a monitored movement and the designated condition is a desired movement.

59. The method of claim 57, wherein the step of delivering includes maintaining a desired position of the monitored structure on a closed loop basis.

60. The method of claim 53, further comprising:
delivering a feedback signal to the patient when the monitored condition corresponds with the designated condition.

61. The method of claim 47, further comprising:
implanting a bioimpedance sensor into the patient;
implanting a passive electrical element into the patient at location away from the bioimpedance sensor;
wherein the step of sensing includes operating the bioimpedance sensor to obtain impedance measurements, including the passive electrical element influencing the obtained impedance measurements.

62. The method of claim 61, wherein the step of implanting a passive electrical element includes locating the passive electrical element at an anatomical structure relevant to at least one of bladder and bowel dysfunction.

63. The method of claim 62, wherein the passive electrical element is located at an anatomical structure relevant to SUI leak causing events.

64. The method of claim 47, wherein the step of sensing includes:
operating a bioimpedance sensor arrangement to emit signals at at least two different frequencies;
obtaining impedance measurements corresponding with each of the at least two different frequencies; and
differentiating between movement of solid tissue and changes in bulk fluid based upon a comparison of the obtained impedance measurements.

65. The method of claim 47, further comprising:
operating at least one bioimpedance sensor implanted within the patient to generate a signal indicative of a condition of a bladder of the patient; and
determining the condition based upon the signal.

66. The method of claim 65, wherein the condition is a fullness of the bladder.
